(19) United States
(12) Patent Application Publication
(10) Pub. No.: US 2019/0282778 A1
(43) Pub. Date: Sep. 19, 2019

(54) CVI THERAPY DEVICE AND METHOD OF USE

(71) Applicant: Kaitlyn Schoen, Old Orchard Beach, ME (US)

(72) Inventor: Kaitlyn Schoen, Old Orchard Beach, ME (US)

(21) Appl. No.: 16/349,172

(22) PCT Filed: Nov. 15, 2017
(86) PCT No.: PCT/US2017/061669

§ 371 Date: May 10, 2019

Related U.S. Application Data
(60) Provisional application No. 62/422,152, filed on Nov. 15, 2016.

(51) Int. Cl. A61M 21/00; G16H 20/30; G09B 19/00; A61N 5/06

(52) U.S. Cl. CPC A61M 21/00; G16H 20/30; G09B 19/00; A61N 2005/0663; A61M 2021/0044; A61M 2205/59; A61N 5/0618

(57) ABSTRACT

The present invention is directed to a therapeutic device that enables the user to effectively treat cortical visual impairment. The therapeutic device comprises a base; a light source within the base capable of generating light in a variety of colors; a light diffusing layer positioned above the light source such that the light diffusing layer is substantially uniform in color when illuminated by the light source; a substantially opaque grid located above the light diffusing layer; an upper light source located above the substantially opaque grid at a sufficient distance to allow physical items to be placed on top of the opaque grip; and the upper light source further positioned such that the upper light source illuminates the upper surface of the substantially opaque grid.

### CROSS-REFERENCE TO RELATED APPLICATIONS

This application claims the benefit of U.S. Provisional Patent Application No. 62/422,152, filed Nov. 15, 2016, the disclosure of which is herein incorporated by reference.

### BACKGROUND

### 1. Field of the Invention

The present invention pertains to the field of therapeutic light boxes. Specifically, the invention relates to a novel therapy device and method that allows individuals with cortical visual impairment (“CVI”) to retrain their brain and develop the connection between sight and feel.

### 2. Discussion of Background Information

Cortical visual impairment is a form of visual impairment that may impact individuals throughout their life. Vision is a vital sense that helps portray communication danger, feelings, environment, opinions, preferences, and many other attributes.

Cortical visual impairment contributes to the vision impairment of many individuals, and prevents the individual from taking part in meaningful occupations and activities of daily living. The primary concern is lack of communication because the appropriate sensory fundamentals are not readily available. A concentrated focus on the development of young, school age children would be highly valued.

Cortical visual impairment typically occurs in infants that experience a premature birth. A premature birth occurs when an infant leaves the placenta, from their mother, before thirty-seven weeks of gestation. Other causes of cortical visual impairment are traumatic brain injury, hypoglycemia, maternal intake of drugs, seizures, and infection to the nervous system.

In premature babies, insufficient growth and development of the child can result in damaged white matter in the brain caused by a lack of blood flow to the neurons and tissue. The brain injury caused by oxygen deprivation to the brain is referred to as hypoxic-ischemic encephalopathy. The damaged periventricular white matter causes impaired visual guidance of movement, deficiency in processing visual motion, and frailty in the comprehension of language, and recognition of objects. These characteristics typically present as lower visual field impairments and they are affiliated with cerebral palsy. Unfortunately, many children who have cortical visual impairment are likely to have another overlapping neurological disability.

Impaired vision affects a great number of occupations as the child progresses through life. An infant with cortical visual impairment cannot engage with their surroundings causing the avenues of communication to close. With difficulty seeing, the child struggles to communicate emotion in response to visual stimuli.

Vision is closely related to communication and movement. A child with cortical visual impairment experiences significant delays in the development of movement, communication, and receipt of information. Receiving information is imperative for a child to indicate danger or create self-expression. Without the ability to see, children with CVI are likely to have delays in their speech and movement may be hindered by the inability to control extremities and core movement. Children with CVI are also likely to become wary of their surroundings, have limited self-expression, and develop a lack of trust in diverse situations.

In a school environment, a child diagnosed with CVI may have difficulty performing the same tasks as another child without CVI. Multi-tasking skills, such as walking and talking simultaneously, tend to be demanding. Some children with CVI become so distressed or angry that they begin to react in violently, while others are restless in classroom settings. In both instances, these behavioral responses can be perceived by peers as bad behavior and without the ability to portray feelings through communication, the child with CVI may accept the false pretenses set by peers. Judgment from peers frequently causes social isolation to develop in the child and the associated withdrawal from social interaction often creates low self-esteem for children in the school environment.

Researchers have discussed the importance of assessing each child through methods such as visual functioning to understand the extent of the CVI. Newcomb, S. “The reliability of the CVI range: A functional vision assessment for children with cortical visual impairment.” Journal of Visual Impairment & Blindness 104 (10) (2010): 637-647. Visual functions assess the skills of the eye—including fixation, tracking, visual fields, and acuity. Visual awareness and acuity are important for deciphering between objects. Object observance is crucial to the fundamentals of children's occupations. Newcomb also notes a response to light and, bright, moving objects in individuals diagnosed with CVI.

Drawing upon the response to light noted by Newcomb, children with CVI can be referred to as “light gazers.” For example, in one study, caregivers were interviewed about children diagnosed with CVI and the authors also observed the light gazing duration of the children. Jan, J. E., M. Groenveld, M., Sykanda, A. M. “Light-Gazing by Visually Impaired Children.” Developmental Medicine and Child Neurology 32, 755-759 (1990). The gazing duration was classified as fifteen seconds or more. During observation, females were considered to be more frequent light gazers and light gazers were predominantly found with children who had congenital CVI compared to children with acquired CVI. Consequently, this evidence provides supplemental proof indicating that both gender and pathology of the damage to the neurons can be further understood and addressed by tapping into the behavior tendencies of light gazers.

By providing children with CVI access to increased light, vibrant colors, and contrasted materials in a classroom environment, the child can develop increased visual acuity. This increased visual acuity leads to an increase of visual inputs, which can increase self-esteem and decrease the communication barrier.

U.S. Patent Application No. 2014/0088439 to Savage discloses an example of an existing light therapy apparatus. The Savage system is comprised of at least one light source that emits above-ambient light at varying intensities and wavelengths. The color of the light varies, depending on the intended purpose. However, the device disclosed by Savage provides the light itself as the source of the therapeutic effect. The device lacks the ability to establish a connection between sight and touch and does not provide the steady, constant, grid-contrasted background of the present invention to retrain the brain of an individual with CVI.

Another example of an existing light therapy device is found in U.S. Patent Application No. 2011/0184498 to Donley. The Donley device is an interactive vision therapy board that includes a display panel with selectively illuminated lights, finger activated switches that are responsive to a patient's touch, and a way to change the light pattern in response to the switches. However, this device is used to enhance eye hand coordination, response/reaction time, and eye tracking, and fails to provide a way for the individual to retrain their brain or to make the make the necessary connections between sight and touch. Further, this device is incapable of treating individuals with CVI using ordinary physical items they encounter during their normal daily activities.

U.S. Pat. No. 5,709,645 to Siever discloses a photic stimulator capable of stimulating the central nervous system. The Siever device is meant to be worn as an eye mask which may be useful for stimulating the nervous system. Again, this system fails to allow the individual to develop the connection between sight and touch. Further, due to the placement of the device directly on the face of the individual, it is impossible to place an ordinary item encountered during normal daily activities between the user and the light source. As a result, the device disclosed in Siever lacks the fundamental ability to train individuals with CVI to recognize important items and to establish the connection between sight and touch that leads to improved social skills and behavioral issues.

While other devices and methods have been proposed for treating CVI, none of the inventions, taken either singly or in combination, adequately address or resolve the aforementioned problems. Therefore, a need exists for a device and method that aids in the treatment of CVI.

### SUMMARY OF THE INVENTION

The present invention solves the problems associated with treating individuals with cortical visual impairment and provides a device and method for effectively treating cortical visual impairment.

The present invention is directed to a therapeutic device that enables the user to effectively treat their cortical visual impairment. The therapeutic device comprises a base; a light source within the base capable of generating light in a variety of colors; a light dispersing layer positioned above the light source such that the light dispersing layer is substantially uniform in color when illuminated by the light source; a substantially opaque grid located above the light dispersing layer; an upper light source located above the substantially opaque grid at a sufficient distance to allow physical items to be placed on top of the opaque grip; and the upper light source further positioned such that the upper light source illuminates the upper surface of the substantially opaque grid.

### BRIEF DESCRIPTION OF THE DRAWINGS

These and other features, aspects and advantages of the present invention will become better understood with reference to the following description, appended claims, and accompanying drawings where:

FIG. 1 is a perspective view of an embodiment of a CVI therapy device of the present invention.

FIG. 2 is an exploded view of an embodiment of a CVI therapy device of the present invention.

FIG. 3 is a perspective view of an embodiment of a CVI therapy device of the present invention.

### DETAILED DESCRIPTION OF THE INVENTION

The present device and method is directed to the problem of treating individuals with cortical visual impairment. Specifically, the present invention provides a device and a method of use that effectively treats cortical visual impairment by retraining the brain to create a connection between what the individual can feel and what the device allows the individual to see.

The present invention addresses the problems surrounding the treatment of cortical visual impairment. Patients and therapists are more likely to see progress when treating cortical visual impairment with a device capable of retraining the brain to make connections between sight and touch. The present invention addresses these needs by providing a device that effectively displays physical items in a manner that allows patients with cortical visual impairment to see them, and a method that allows the patient to physically manipulate the physical item before, during or after treatment with the device.

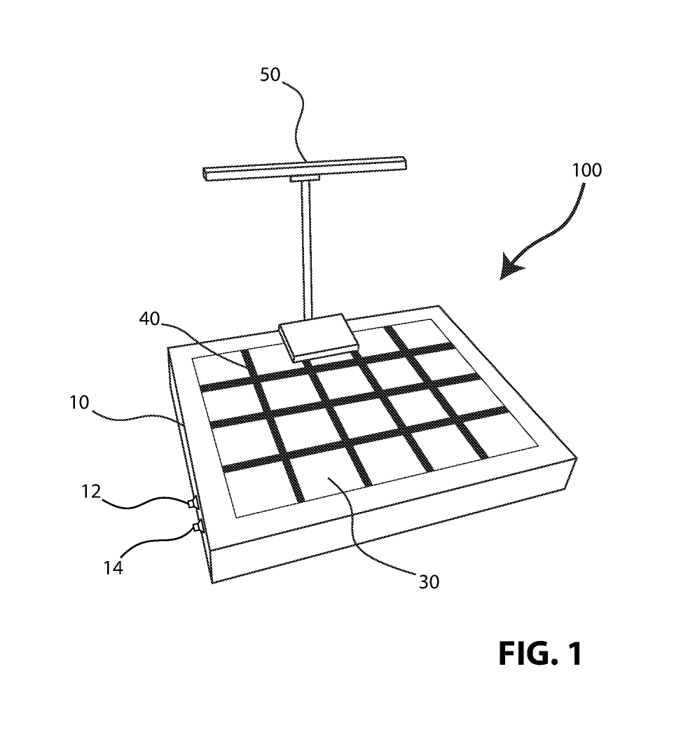

Turning to FIG. 1, a CVI therapy device 100 of the present invention is shown. The CVI therapy device 100 includes a base 10, which is placed below a light diffusing layer 30. A grid 40 is placed on top of the light diffusing layer 30. In some embodiments, the grid 40 functions as the upper surface of the CVI therapy device 100.

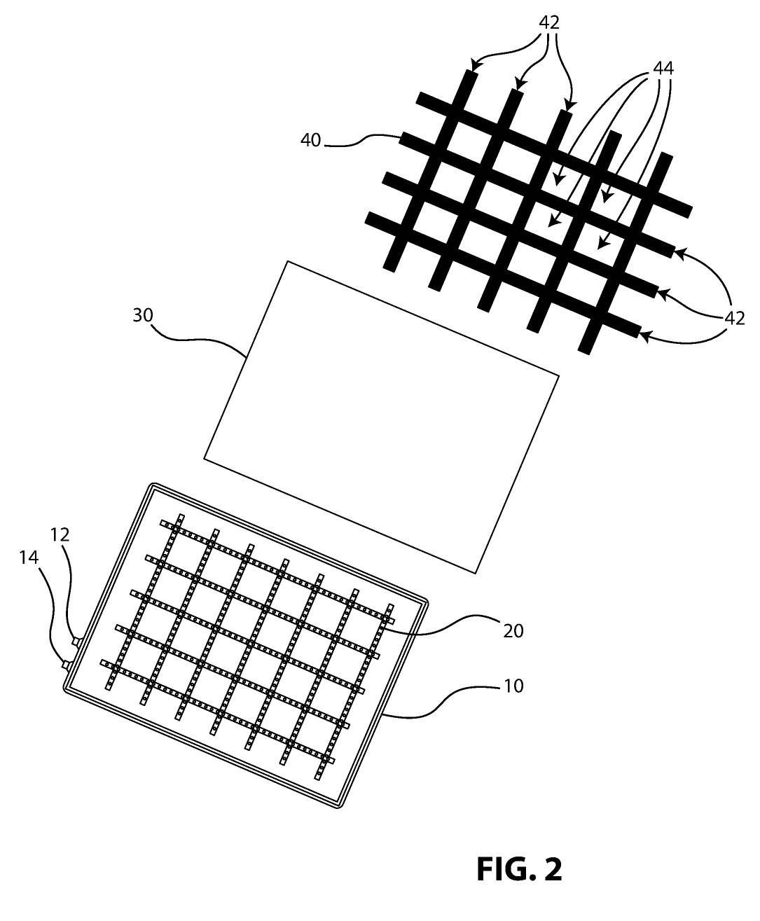

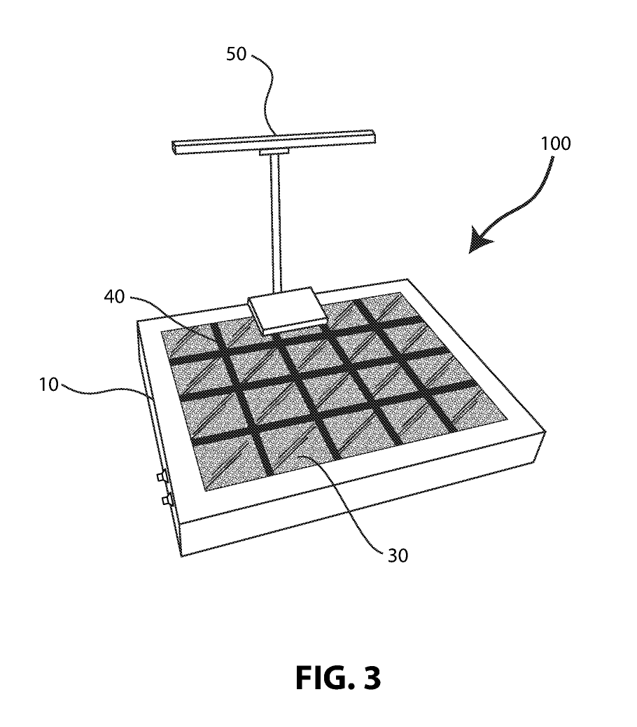

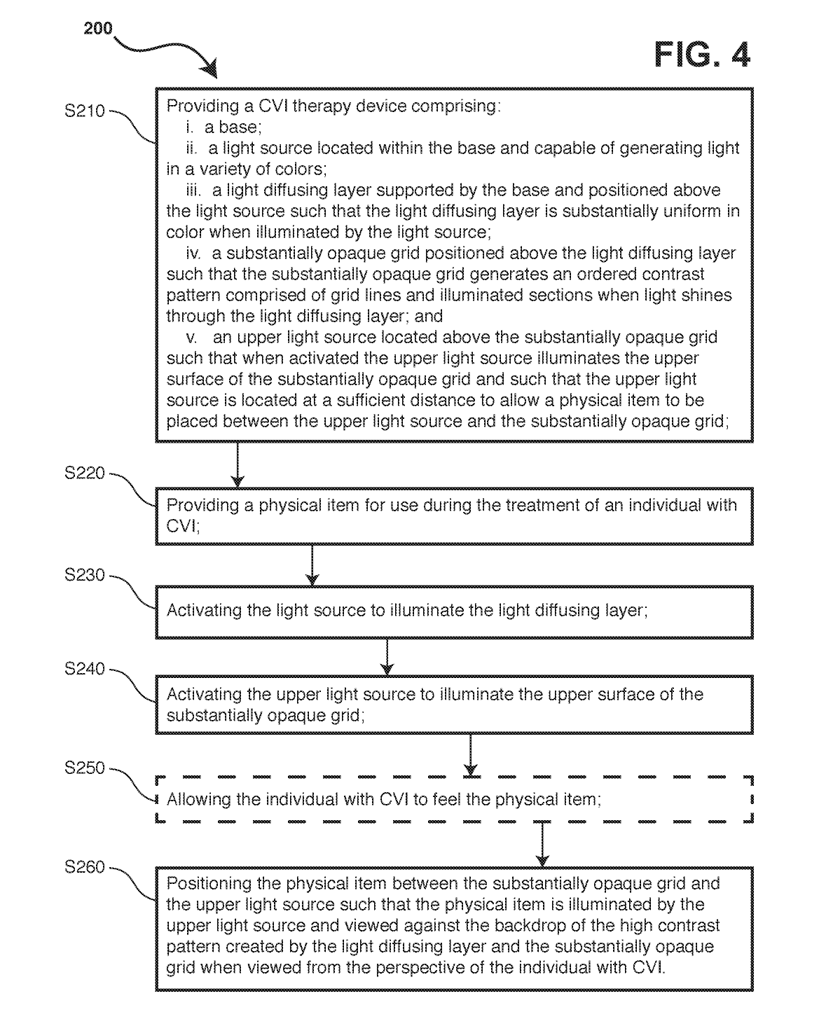

With reference to both FIGS. 1 and 2, the base 10 is sized and shaped to accommodate a light source 20 and to support the light diffusing layer 30 and the grid 40. In addition, the base 10 must be large enough in length and width to effectively provide a background for the applicable physical items that are placed on the upper surface of the CVI therapy device 100. The base 10 is preferably substantially opaque to ensure that the light generated by the light source 20 is emitted through the light diffusing layer 30 toward the patient receiving treatment.

The base 10 may have a solid bottom or the bottom of the base 10 may be open. In situations where the bottom of the base 10 is left open, it is preferable to locate the CVI therapy device 100 on a table or other flat surface to ensure that light emitted from the base 10 is directed toward the patient receiving therapy.

The inside of the base 10 may be lined with reflective material, with a light source 20 attached within, in order to create a substantially uniform light. The base 10 may include a slot for inserting a removable grid 40.

Located inside the base 10 is a light source 20. The light source 20 may be a single light directed toward the light diffusing layer 30 or, as depicted in FIG. 2, the light source 20 may be comprised of a plurality of lights each directed to a portion of the light diffusing layer 30. Where the light source 20 is comprised of a plurality of lights, the plurality of lights may be arranged in any fashion, provided they sufficiently illuminate the light diffusing layer 30. For example, the plurality of lights may be arranged in a grid pattern as depicted in FIG. 2, they may be arranged in rows placed either vertically or horizontally within the base 10, or they may be strategically placed throughout the base 10. In addition, the base 10 may be adapted to improve the efficiency of the light source 20. For example, the interior surface of the base 10 may include reflective material to evenly distribute or to focus the light toward the light diffusing layer 30.

The light source 20 may be capable of generating only white light or the light source 20 may be capable of a generating any wavelength on the visible light spectrum, selectable by the user or the therapist. The light source 20 is capable of generating a range of colors so that the therapist can tune the light source 20 to the wavelength of visible light that provides the best response in the patient receiving treatment. For example, one patient, may respond better to an orange or red hue in the range of about 590 nm to about 700 nm, while a second patient may respond better to a yellow hue in the range of about 570 nm to about 590 nm.

As depicted in FIG. 2, a first knob 12 can be used as a control to adjust the wavelength of the light generated by the light source 20. In addition to controlling the wavelength of the light generated by the light source 20, a second knob 14 may also be included to adjust the brightness of the light emitted by the light source 20. While knobs are depicted in FIG. 2, it should be understood that the wavelength and brightness adjustment of the light source 20 may be controlled by any of the controls known within the art. For example, any form of knob or switch capable of selectively adjusting the light source 20 could be used.

Preferably, controls are provided to separately adjust the wavelength of the light generated by the light source 20 and the brightness of the light generated by the light source 20. However, the adjustment of wavelength and brightness may be combined into a single control or even programmed in order to quickly adjust to a specific color and specific brightness.

Above the light source 20, and supported by the base 10, is a light diffusing layer 30. The light diffusing layer 30 ensures that a substantially uniform lighting pattern is projected to the patient receiving treatment. In embodiments where the light source 20 is comprised of a plurality of lights, the individual lights may each generate slightly different color light. In such situations, the light diffusing layer 30 ensures that a substantially uniform color pattern is presented to the patient receiving treatment. Preferably, the light diffusing layer 30 ensures that the color and brightness of the light diffusing layer 30 may be made of any suitable form of glass or plastic as known in the art.

In embodiments where the base 10 includes reflective or diffusing elements that disperse the light generated by the light source 20 before the light passes through the light diffusing layer 30, the light diffusing layer 30 may be essentially transparent, since there is little need to diffuse the light further to create a substantially uniform light pattern. However, in situations where the light is directed directly at the light diffusing layer 30, the light diffusing layer 30 may be frosted or otherwise configured to sufficiently diffuse the light and generate a substantially uniform light pattern. In addition, the light diffusing layer 30 may be a single layer or it may be a plurality of layers oriented to achieve the desired level of diffusion.

Positioned directly above and preferably in contact with the light diffusing layer 30 is the grid 40. The grid 40 includes grid lines 42, which can be any opaque or substantially opaque material. The grid 40 is positioned above the light diffusing layer 30 to block a portion of the light generated by the light source 20 in order to create an ordered contrast pattern comprised of unlit grid lines 42 and illuminated sections 44. The grid 40 may form the upper surface of the CVI therapy device 100 or the grid 40 may be positioned below one or more substantially transparent layers. Where one or more substantially transparent layers are located above the grid 40, the outer surface of the one or more substantially transparent layers forms the upper surface of the CVI therapy device 100.

Further, the grid 40 may be fixed in place or may be removable, and the dimensions of the grid 40 may vary without deviating from the scope of the present invention. Indeed, it may be desirable to vary the dimensions of the grid 40 according to the needs of the patient receiving treatment and the desired size of the physical items being used during treatment. Varying the width of the grid lines 42 created by the grid 40 will adjust the separation between the unblocked areas of light when the CVI therapy device 100 is viewed during treatment. The grid 40 may be configured to generate a contrast pattern with grid lines 42 as thin as about 0.125 inches up to as wide as about 2 inches. In addition, the spacing between grid lines 42 created by the grid 40 will determine the size of the illuminated sections 44 visible to the patient receiving treatment. The grid 40 may be configured to generate illuminated sections 44 as small as about 1-inch square up to a size of about 5-inch square.

Throughout the course of treatment, the therapist may require a wider or narrower grid pattern, depending on how advanced the patient is in their treatment or the physical item being used. Embodiments where the grid 40 is removable facilitate the customization of the CVI therapy device 100 to the needs of each individual patient. Where the grid 40 is removable, the grid 40 may be supported as known in the art to facilitate exchange of various grid 40 configurations. For example, the grid 40 may rest directly on top of the light diffusing layer 40 or the base 10 or the base 10 may include a slot or other means for the grid 40 to slide into place.

Located above the upper surface of the grid 40, an upper light source 50 provides additional assistance throughout the course of the therapy. Some individuals affected by CVI may require more light than the CVI therapy device provides in order to focus and may benefit from an upper light source 50. An upper light source 50 may be used selectively to enhance the effectiveness of the CVI therapy device 100 by providing additional illumination of the physical item on an as needed basis. The upper light source 50 may be attached to the base 10 or it may be a separate overheard light source. Where the upper light source 50 is attached to the base 10, the upper light source 50 may be attached to the base 10 using a fixed or adjustable arm. In addition, the upper light source 50 may include a switch capable of independently activating the upper light source 50. The switch may be an adjustable switch, capable of adjusting the brightness of the upper light source 50.

Preferably, the upper light source 50 generates traditional white light to illuminate the physical item being displayed during treatment. However, in some embodiments the upper light source 50 is capable of generating a range of colors. In such embodiments, a separate control means, such as a knob, switch or other known control device, may be included to adjust the wavelength of the light generated by the upper light source 50. Alternatively, the color of the upper light source 50 and the color of the light source 20 may be adjusted using a single control means capable of simultaneously adjusting the color output of the light source 20 and the upper light source 50.

A method of the present invention includes a method for treating a patient with CVI using a CVI therapy device 100. The method includes providing a CVI therapy device 100 and a physical item. The physical item may be any physical item known to a person of skill in the art as being useful for the treatment of CVI. While physical items are usually small and can include items such as a marker, doll or toy car, the specific nature of the physical items may vary depending on factors such as age, extent of neurological damage, and current comprehension and vocabulary.

The CVI therapy device 100 is adjusted so that the background displays a desired color at a desired brightness. Where the CVI therapy device 100 includes control features, the color and brightness of the light source 20 can be tailored to the patient receiving treatment. Once the CVI therapy device 100 is on and at the correct color and brightness, the physical item is placed on the upper surface of the CVI therapy device 100 such that when the patient receiving treatment views the physical item it is positioned above the grid 40. The patient then views the physical item for a specified duration of time, which is dependent on each individual patient's occupational needs. The brightness of the light source 20 may be adjusted throughout treatment.

In many instances, manipulation of the physical item will improve the efficacy of the treatment using the disclosed method. Therefore, as an optional step, the patient receiving treatment may hold, manipulate, or otherwise touch the physical item prior to the placement of the physical item on the CVI therapy device 100. In addition, because the physical item is present in front of the patient, the patient may interact with the physical item during treatment by touching the physical item as it rests on the CVI therapy device 100 or picking up the physical item and removing it from the CVI therapy device 100.

Additionally, the method of the present invention may include activating the upper light source 50 such that the upper light source 50 illuminates the upper surface of the physical item. The upper light source 50 may be activated prior to beginning treatment with the CVI therapy device 100 or may be selectively activated as required during treatment to increase brightness and subsequently modify color.

Turning to FIG. 4, a method 200 for using a CVI therapy device 100 to treat individuals with CVI is depicted. A first step S210 comprises providing a CVI therapy device 100 and a second step S220 comprising providing a physical item for use during treatment of an individual with CVI. A third step S230 comprises activating the light source 20 to illuminate the light diffusing layer 30, while a fourth step S240 comprises activating the upper light source 50 to illuminate the upper surface of the substantially opaque grid 40. An optional fifth step S250 comprises allowing the individual with CVI to feel the physical item prior to performing a sixth step S260, comprising positioning the physical item between the substantially opaque grid 40 and the upper light source 50 such that the physical item is illuminated by the upper light source 50 and viewed against the backdrop of the high contrast pattern created by the light diffusing layer 30 and the substantially opaque grid 40 when viewed from the perspective of the individual with CVI.

It is noted that the foregoing examples have been provided merely for the purpose of explanation and are in no way to be construed as limiting of the present invention. While the present invention has been described with reference to exemplary embodiments, it is understood that the words, which have been used herein, are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A CVI therapy device comprising:
a. a base;
b. a light source located within the base and capable of generating light in a variety of colors;
c. a light diffusing layer supported by the base and positioned above the light source such that the light diffusing layer is substantially uniform in color when illuminated by the light source;
d. a substantially opaque grid positioned above the light diffusing layer such that the grid generates an ordered contrast pattern comprised of grid lines and illuminated sections when light shines through the light dispersing layer;
e. an upper light source located above the substantially opaque grid such that when activated the upper light source illuminates the upper surface of the substantially opaque grid and such that the upper light source is located at a sufficient distance to allow a physical item to be placed between the upper light source and the substantially opaque grid.

2. The CVI therapy device of claim 1 further comprising a first knob for adjusting the wavelength of the light generated by the light source.

3. The CVI therapy device of claim 2 further comprising a second knob to adjust the brightness of the light emitted by the light source.

4. The CVI therapy device of claim 1 wherein the wavelength of the light generated by the light source is in the range of about 590 nm to about 700 nm.

5. The CVI therapy device of claim 1 wherein the substantially opaque grid is removable.

6. The CVI therapy device of claim 1 wherein the thickness of the grid lines is in the range of about 0.125 inches to about 2 inches.

7. The CVI therapy device of claim 1 wherein the size of the illuminated sections is in the range of about 1-inch square to about 5-inches square.

8. A method for treating individuals with CVI comprising:
a. providing a CVI therapy device comprising:
i. a base;
ii. a light source located within the base and capable of generating light in a variety of colors;
iii. a light diffusing layer supported by the base and positioned above the light source such that the light diffusing layer is substantially uniform in color when illuminated by the light source;
iv. a substantially opaque grid positioned above the light diffusing layer such that the substantially opaque grid generates an ordered contrast pattern comprised of grid lines and illuminated sections when light shines through the light diffusing layer; and
v. an upper light source located above the substantially opaque grid such that when activated the upper light source illuminates the upper surface of the substantially opaque grid and such that the upper light source is located at a sufficient distance to allow a physical item to be placed between the upper light source and the substantially opaque grid
b. providing a physical item for use during treatment of an individual with CVI;
c. activating the light source to illuminate the light diffusing layer;
d. activating the upper light source to illuminate the upper surface of the substantially opaque grid;
e. allowing the individual with CVI to feel the physical item;
f. positioning the physical item between the substantially opaque grid and the upper light source such that the physical item is illuminated by the upper light source and viewed against the backdrop of the high contrast pattern created by the light diffusing layer and the substantially opaque grid when viewed from the perspective of the individual with CVI.

9. The method for treating individuals with CVI of claim 8 wherein the provided CVI therapy device further comprises a first knob for adjusting the wavelength of the light generated by the light source.

10. The method for treating individuals with CVI of claim 9 wherein the provided CVI therapy device further comprises a second knob to adjust the brightness of the light emitted by the light source.

11. The method for treating individuals with CVI of claim 10 wherein the brightness of the light emitted by the light source is adjusted prior to positioning the physical item between the substantially opaque grid and the upper light source.

12. The method for treating individuals with CVI of claim 8 wherein the wavelength of the light generated by the light source is in the range of about 570 nm to about 590 nm.

13. The method for treating individuals with CVI of claim 8 wherein the wavelength of the light generated by the light source is in the range of about 590 nm to about 700 nm.

14. The method for treating individuals with CVI of claim 8 wherein the item positioned in Step e is positioned on the upper surface of the CVI therapy device.

15. The method for treating individuals with CVI of claim 8 wherein the substantially opaque grid is removable.

16. The method for treating individuals with CVI of claim 8 wherein the thickness of the grid lines is in the range of about 0.125 inches to about 2 inches.

17. The method for treating individuals with CVI of claim 8 wherein the size of the illuminated sections is in the range of about 1-inch square to about 5-inches square.

18. The method for treating individuals with CVI of claim 8 wherein the physical item is selected from the group consisting of a marker, a doll, and a toy car.
